(19) United States
(12) Patent Application Publication
(10) Pub. No.: US 2010/0249891 A1
(43) Pub. Date: Sep. 30, 2010

(54) IMPLANTABLE APPARATUS FOR THE TREATMENT OF A SURFACE OF A DAMAGED VESSEL OR BODY CAVITY BY ELECTROMAGNETIC ENERGY

(75) Inventors: DAVID O'FLYNN, Bray (IE); J. Todd Derbin, Solebury, PA (US); Roni Appel, Caesarea (IL); Dymphna Donaghy, Letterkenny (IE)

Correspondence Address: FULWIDER PATTON LLP, HOWARD HUGHES CENTER, 6060 CENTER DRIVE, TENTH FLOOR, LOS ANGELES, CA, 90045, US

(73) Assignee: Arista Therapeutics, Inc., Solebury, PA (US)

(21) Appl. No.: 12/611,927

(22) Filed: Nov. 3, 2009

Related U.S. Application Data
(60) Provisional application No. 61/163,818, filed on Mar. 26, 2009.
(60) Provisional application No. 61/165,468, filed on Mar. 31, 2009.
(60) Provisional application No. 61/165,858, filed on Apr. 1, 2009.

(51) Int. Cl. A61N 5/06; A61F 2/82

(52) U.S. Cl. 607/92; 623/1.15

(57) ABSTRACT

The present invention provides an improved apparatus and method for the treatment of body cavities and damaged vessels using electromagnetic energy. A device and apparatus according to the invention may be used to irradiate a tissue surface internal to the body, for example, for treatment of an aneurysm, tissue reconstructing, or removal of an anomaly in a blood vessel. Light energy may be radiated from an implantable and retrievable biocompatible matrix into which is mounted a plurality of electromagnetic energy sources, such as light emitting diodes or the like.

### CROSS-REFERENCES TO RELATED APPLICATIONS

This application claims priority from U.S. Application No. 61/163,818 filed on Mar. 26, 2009, U.S. Application No. 61/165,468 filed on Mar. 31, 2009, and U.S. Application No. 61/165,858 filed on May 1, 2009, each of which are incorporated by reference in their entirety.

### BACKGROUND

This invention relates to medical devices and more specifically to such devices for the treatment of body cavities and damaged vessels using electromagnetic energy.

Application of electromagnetic energy to a tissue surface has been used in several medical treatments. For example, it is known to apply light to a tissue surface in order to heal a pathological state, to remove a stenosis in a blood vessel or for laser welding of tissues, for example in order to treat a rupture in a vessel wall or to perform an anastomosis of two blood vessels. It is also known to use application of electromagnetic energy for tissue regeneration and therapy. For example, low level laser irradiation in the visible to infrared range of the light spectrum has been clinically shown to accelerate healing in skin wounds, and reduce pain and inflammation in musculoskeletal disorders. The underlying mechanisms are initiating (biostimulating) processes such as collagen synthesis, cell proliferation, and reducing secretion of inflammatory markers. Gavish et al., Lasers in Surgery and Medicine (2006) 38:779-786, which is incorporated herein by reference, discloses that low level laser in vitro stimulates vascular smooth muscle cell proliferation and collagen synthesis, modulates the equilibrium between regulatory matrix remodeling enzymes, and inhibits pro-inflammatory IL-1-β gene expression.

An apparatus has been described for applying electromagnetic energy to the heart tissue for a biostimulative and cytoprotective effect. An apparatus to provide electromagnetic biostimulation of tissue, which includes a source of electromagnetic radiation and optics operatively connected to the source of electromagnetic radiation, can be used for directing electromagnetic radiation from the outside of the body to the tissue surface.

Additionally, low energy light exposure has been found to both inhibit restenosis following dilation of a stenotic region, and to inhibit vascular spasms, whether or not they are associated with a stenotic region. Such light energy has also been found to arrest progress of various types of a stenosis and expose a vessel wall to light energy from an intravascular approach for the prevention of restenosis.

It is also known that an apparatus may be used for applying light to the interior surface of a vascular wall for laser treatment of the vessel. Light may be generated by an extracorporeal light source guided by a light guide to the interior of the blood vessel to be treated. A light deflector and diffuser may be used to direct the light in a substantially radial fashion onto the vessel wall.

Abdominal Aortic Aneurysm (AAA) formation is an arteriosclerotic process characterized by marked disruption of the musculoelastic lamellar structure of the media. Rupture of an untreated AAA is particularly life threatening. Extensive destruction of the elastic tissue is associated with marked inflammatory cell infiltration and progressive diminution in the number of viable smooth muscle cells. Over time, and aggravated by contributory risk factors such as systolic hypertension, aneurysm growth occurs through a complicated, but insidious, imbalance between matrix protein production and degradation, favoring expansion, thereby increasing the risk of rupture of the weakened wall.

AAA is present in approximately 10% of individuals over the age of 65 years, with its frequency increasing as the proportion of elderly individuals in the general population continues to rise. It is widely known that the risk of rupture increases in approximate proportion to aneurysm size, which can be monitored by computed tomography (CT), ultrasound, or magnetic resonance imaging (MRI). The estimated risk of rupture ranges from 10-20% for an abdominal aneurysm 6-7 cm in diameter, to 30-50% if the maximum diameter is greater than 8 cm. Overall mortality from a ruptured AAA is greater than 90%. Current forms of aneurysm treatment focus either on the open abdomen, surgical, graft-based repair or endovascular exclusion of the diseased segment of aorta with large, membrane-covered, e.g. Gortex covered stents. Both techniques have major side effects with potentially life-threatening consequences, particularly in patients of advanced age (the majority of patients) or others at high risk or compromised cardiac function.

Gertz et al. WO 2007/113834, which is incorporated herein by reference, discloses a device and method for illuminating a tissue surface. In Gertz, a light source is optically coupled to the proximal end of a light guide and a light scatterer is optically coupled to the distal end of the light guide. The device includes a deployment mechanism that is configured to bring the light scatterer from an undeployed small caliber configuration in which the light scatterer is delivered to the body surface to a deployed large caliber configuration in which the light scatterer irradiates the body surface.

It is known that biocompatible material may be a polymeric material such as PET fabric or porous PTFE. Alternatively, the biocompatible material may be a material of biological origin such as harvested bovine or human blood vessels. The biocompatible material may also be made of conventional vascular graft materials such as GORE-TEX®.

While the techniques described in the above references describe generally the benefits of the techniques and methods for using the electromagnetic spectrum to treat tissue surfaces, the use of a remote light source and optical paths can create difficulties in the in vitro use of the processes and apparatus in the prior art, such as localized heating, and are not intended to be placed for long periods of time to vary the form and energy of treatment of the tissue. Accordingly, the present invention provides an implantable biocompatible apparatus for the treatment of an interior surface of a damaged vessel or internal body cavity by electromagnetic energy.

### SUMMARY OF THE INVENTION

The present invention provides an improved apparatus and method for the internal treatment of internal body cavities and damaged internal vessels using electromagnetic energy. A device and apparatus according to the invention may be used to irradiate an internal tissue surface, for example, for treatment of an aneurysm, tissue reconstructing, or removal of an anomaly in a blood vessel. Light energy may be radiated from an implantable and retrievable biocompatible matrix into which is mounted a plurality of electromagnetic energy sources, such as light emitting diodes (LED's), with the appropriate output frequency.

In a presently preferred embodiment, the biocompatible material used to make the matrix may be a polymeric material such as polyethylene terephthalate (PET) or porous polytetrafluoroethylene (PTFE). Alternatively, the biocompatible material may be a material of biological origin such as material harvested or grown from bovine or human blood vessels. The biocompatible material may also be made of conventional vascular graft materials such as GORE-TEX®. The base substrate of the biocompatible material may be preferably a porous, non-fabric substrate such as porous expanded PTFE.

In a presently preferred embodiment, the biocompatible of the invention includes a reinforcing carrier backing attached to an LED-embedded matrix. Activation and control of the energy emitted by the LED's may be remotely controlled by electrical connections to a remote electrical source, whereby the electrical connections form part of the placement system, such as a guiding catheter. A programmable energy supply may be used to supply the light sources in the apparatus with the energy via electrical connections through a catheter. Alternatively, activation and control of the electromagnetic energy emitted may be achieved through RF coupling from an external source to an internal cavity. The programmability of external RF or electrical energy source allows for the control and variation of the radiation therapy used, and the radiation frequency at which it is applied without the requirement to move or alter the coverage of the light emitting devices.

In a presently preferred embodiment, the implantable illuminating biocompatible composite matrix may be used in conjunction with known endovascular placement systems, including those used to place vascular grafts and other implantable devices which are compatible with malformations or weakened arterial structures to be treated.

In a further aspect of a presently preferred embodiment, the implantable illuminating biocompatible matrix may be used in conjunction with known endovascular grafts and other implantable devices that are compatible with malformations or weakened body structures to be treated and adapted to the special purposes of the invention.

Other features and advantages of the present invention will become more apparent from the following detailed description of the preferred embodiments in conjunction with the accompanying drawings, which illustrate, by way of example, the operation of the invention.

### BRIEF DESCRIPTION OF THE DRAWINGS

FIG. 1 illustrates an enlarged perspective view of an apparatus according to one embodiment of the invention for irradiating an internal body cavity;

FIG. 2 illustrates an apparatus for irradiating an internal body cavity in accordance with a second embodiment of the invention which may be placed on the external surface of a stent or other placement substrate used in a body cavity; and

FIG. 3 illustrates a cross-sectional view at 3-3 of the apparatus of FIG. 2 of an embodiment of the invention.

### DETAILED DESCRIPTION OF THE PREFERRED EMBODIMENTS

The present invention provides an implantable biocompatible medical apparatus generally indicated by 10, for the treatment of internal body cavities and damaged vessels using electromagnetic energy. Electromagnetic energy is irradiated from a biocompatible matrix into which is embedded a plurality of light sources.

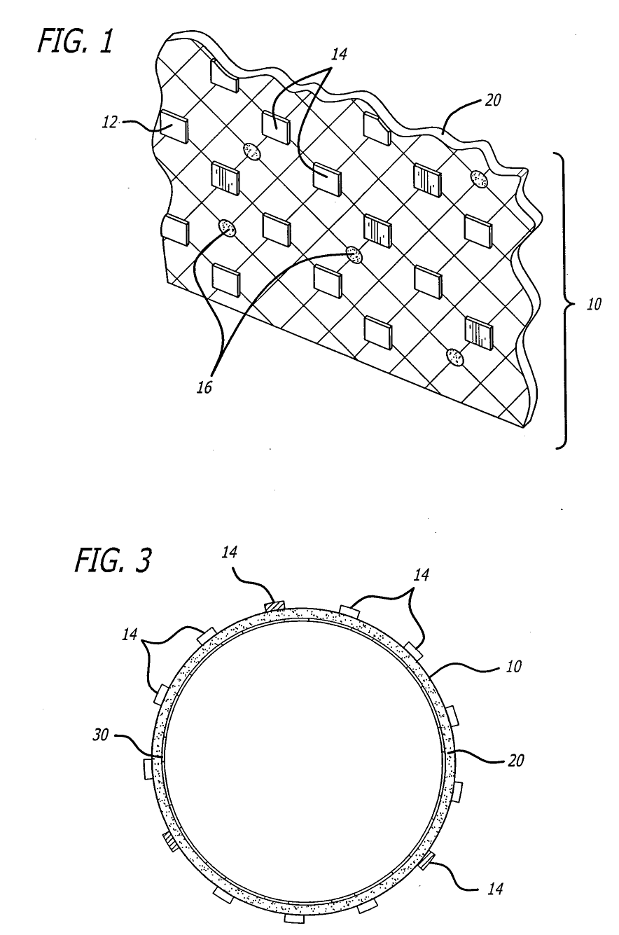

FIG. 1 illustrates an apparatus for irradiating an internal body surface to be treated in accordance with one embodiment of the invention. The illuminating device 10 comprises a biocompatible matrix 12 into which multiple LED's 14 are embedded and onto which is secured a reinforcing carrier sleeve 20. The illuminating device with the reinforced matrix is a sterile apparatus.

In one presently preferred embodiment, a biocompatible matrix 12 into which is embedded a plurality of LEDs 14 and onto which is secured a reinforcing carrier sleeve 20, is placed on the surface of a body cavity to be treated via a catheter or the like. The reinforced matrix 12 preferably covers a substantial portion of the interior surface of the body cavity to be treated, and as such, is reinforced by the backing material. The reinforced matrix may be constructed from flexible and biocompatible materials, some of which may have shape memory characteristics. In a presently preferred embodiment, the biocompatible material used to make the matrix may be a polymeric material such as PET or porous PTFE. Alternatively, the biocompatible material may be a material of biological origin such as material harvested or grown from bovine or human blood vessels. The biocompatible material may also be made of conventional vascular graft materials such as GORE-TEX®. The base substrate of the biocompatible material may be preferably a porous, non-fabric substrate such as porous expanded PTFE. The biocompatible material may also include Nitonol, polyurethane, and Teflon.

Once the reinforced matrix 12 is deployed to the desired location, the device of the present invention (which may be retrieved post-implantation), and tissue surface to be treated will be irradiated through electromagnetic energy generated by the embedded light sources 14. Essentially the entire surface area of the body cavity which is in contact with the irradiating surface of the reinforced matrix is available to be irradiated. Additionally, the surface treatment can be varied according to desired treatment schedules by time phasing of the energy from the light source.

In an additional aspect of the invention, the reinforced matrix may have circumferential rigidity to assist in the prevention of rupture of the vessel or body cavity during treatment, and to provide more structure for the light sources.

The irradiating device of the invention may be provided with a means for firmly attaching the surface of the reinforced matrix to the tissue surface to be treated. Such means may comprise use of an adhesive surgical glue 16, for example, a cyanoacrylate, bioglue, or fibrin-type glue. The adhesive surgical glue of the present invention is biocompatible. The adhesive may be used on the side of the reinforced matrix which will be anchored to the tissue surface being treated. The other side of the reinforced matrix will preferably not contain adhesive material so as to prevent the LEDs from being covered and/or functionally impacted in a negative manner. Use of surgical glue is useful to hold the reinforced matrix in place until tissue in-growth through the matrix occurs. This avoids the necessity, for instance, of wrapping the matrix around the entire external surface of the reinforcing matrix inside the body cavity, if that is the desired treatment option.

In another embodiment of the present invention, a combination of both biodegradable and non-biodegradable materials may be used in constructing the reinforced matrix of the invention, in order to allow the device to be implanted and secured while also maintaining the ability to remove at least a portion of the device when treatment has been completed.

The electromagnetic energy source is selected in accordance with the requirements of the particular application. For example, in order to treat a damaged wall of an artery such as an aneurysm, low level laser irradiation (also known as “low energy laser”, “photo-biostimidation” and “red-light therapy”) in the range of 500 to 900 nanometers (nm), and more preferably in the range of 600 to 900 nm, may be used that is preferably emitted from the illuminating surface with an energy flux in the range of about 0.01 to about 50 Joules/cm², and more preferably from about 0.1 to about 5 Joules/cm². For surgical welding, light in the 780 to 2010 nm range may be used, in which case the light source may be a semiconductor diode laser that generates 808 nm light or a diode-pumped Ho: Y AG laser which generates 2010 nm light.

In another aspect of the invention, the present invention provides a method for treating a tissue surface. In accordance with this aspect of the invention, the illuminating device of the invention is delivered with the electromagnetic energy illuminating surface to the body site to be treated. The illuminating surface is then applied to the surface to be treated, and the surface to be treated is radiated.

In an additional aspect of the invention, the implantable biocompatible matrix of the present invention may be used to irradiate the perivascular surface of a damaged blood vessel, for example, in order to treat an aneurysm by arresting its progression and growth. Once implanted, the irradiating elements of the illuminating device will radiate in the direction of the area to be treated.

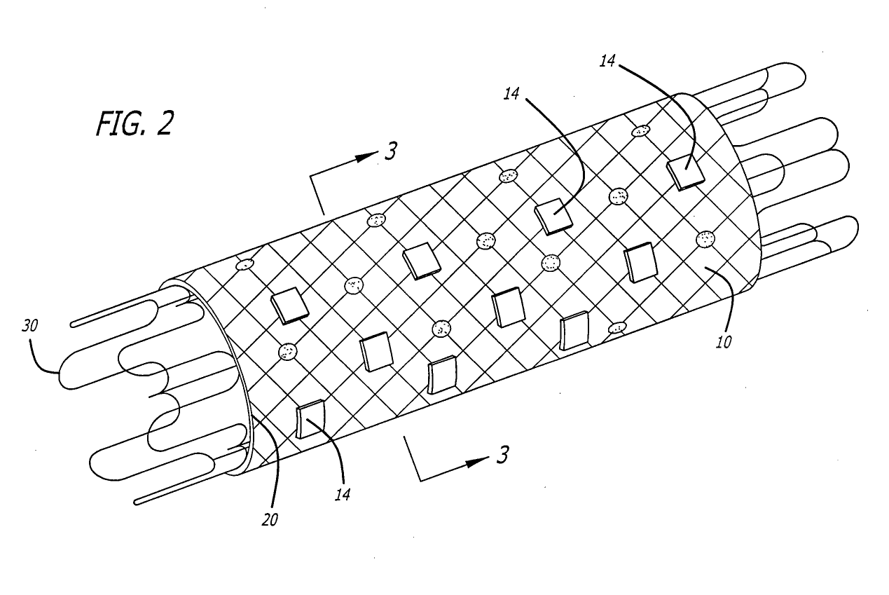

FIG. 2 shows the illuminating device 10, in accordance with another embodiment of the invention. As shown in the figure, the implantable illuminating biocompatible 12, into which are embedded a plurality of light sources 14, may be used in conjunction with well known endovascular placement systems 30, including those used to place endovascular stents and other implantable devices that are compatible to malformations needing treatment. In a presently preferred embodiment, the biocompatible 12 may be mounted on the exterior surface of an endovascular stent, laproscopic device, catheter, or other implantable device compatible with the treatment of malformations and placed in the desired area to be treated. Use of the endovascular placement system 30 in conjunction with the illuminating device 10 and the reinforced matrix, will provide additional mechanical strength and stability to the internal body cavity to be treated and will enable the delivery of the biocompatible and illuminating apparatus through a well established lower risk medical procedure.

In an additional embodiment of this invention, after the implantable device is utilized to irradiate the tissue surface to be treated, the device may be fully or partially removed. The device may be removed after a defined treatment time pattern depending determined on a case-by-case basis by the administering physician. In one embodiment, the cushion layer of LEDs may be removed and the biocompatible matrix would be left adhered to the tissue surface being treated. In an alternative embodiment, the entire illuminating device with its reinforced matrix may be surgically removed. If full removal of the device is necessary, there may be a need for surgical removal because the tissue surface being treated may have experienced tissue growth through the matrix.

FIG. 3 depicts a cross-sectional view of the illuminating device 10 of FIG. 2 at 3-3, with the biocompatible matrix 12, LEDs 14, reinforcing backing layer 20, and stent 30 as shown.

In another preferred embodiment, the invention may also be configured such that a biocompatible matrix is designed to be left in place as an endovascular or intravascular reinforcement after treatment, either with or without the additional layer of electromagnetic emitters remaining in place.

It will be apparent from the foregoing that while particular forms of the invention have been illustrated and described, various modifications can be made without departing from the spirit and scope of the invention. Accordingly, it is not intended that the invention be limited, except as by the claims.Text

We claim:

1. An implantable apparatus for internal treatment of body cavities and damaged internal vessels using electromagnetic energy, comprising:
a biocompatible matrix including an adhesive for attaching the reinforced matrix to a tissue surface to be treated;
a plurality of electromagnetic energy sources mounted to said biocompatible matrix; and
a remote electrical source in electrical communication with said plurality of electromagnetic energy sources for activation and control of energy emitted by said electromagnetic energy sources.

2. The implantable apparatus of claim 1, wherein said adhesive is selected from the group consisting of surgical glue, a cyanoacrylate adhesive, bioglue, and a fibrin-type glue.

3. The implantable apparatus of claim 1, further comprising a reinforcing carrier backing attached to said biocompatible matrix.

4. The implantable apparatus of claim 3, wherein said reinforcing carrier backing comprises a reinforcing carrier sleeve.

5. The implantable apparatus of claim 3, wherein said matrix and reinforcing carrier backing in combination has circumferential rigidity.

6. The implantable apparatus of claim 1, wherein said biocompatible matrix is formed from a polymeric material.

7. The implantable apparatus of claim 6, wherein said polymeric material is selected from the group consisting of a porous, non-fabric substrate, PET fabric, porous PTFE, and porous expanded PTFE.

8. The implantable apparatus of claim 1, wherein said biocompatible matrix is formed from a material of biological origin.

9. The implantable apparatus of claim 8, wherein said material of biological origin is selected from the group consisting of material harvested or grown from bovine or human blood vessels.

10. The implantable apparatus of claim 1, wherein said biocompatible matrix is formed from a vascular graft material.

11. The implantable apparatus of claim 10, wherein said vascular graft material comprises a porous, non-fabric substrate.

12. The implantable apparatus of claim 1, wherein said plurality of electromagnetic energy sources comprise a plurality of light sources.

13. The implantable apparatus of claim 1, wherein said plurality of electromagnetic energy sources comprise a plurality of light emitting diodes embedded in said biocompatible matrix.

14. The implantable apparatus of claim 1, wherein said plurality of electromagnetic energy sources provide irradiation in the range of 500 to 2010 nm.

15. The implantable apparatus of claim 1, wherein said plurality of electromagnetic energy sources provide low level laser irradiation.

16. The implantable apparatus of claim 14, wherein said irradiation is in the range of 500 to 900 nm.

17. The implantable apparatus of claim 14, wherein said irradiation is in the range of 780 to 2010 nm.

18. The implantable apparatus of claim 14, wherein said irradiation is in the range of 600 to 900 nm.

19. The implantable apparatus of claim 14, wherein said irradiation has an energy flux in the range of about 0.01 to about 50 Joules/cm².

20. The implantable apparatus of claim 14, wherein said irradiation has an energy flux in the range of about 0.1 to about 5 Joules/cm².

21. The implantable apparatus of claim 1, wherein said remote electrical source comprises at least one electrical connection to the remote electrical source.

22. The implantable apparatus of claim 1, wherein said remote electrical source comprises a programmable energy supply to supply electrical energy to the electromagnetic energy sources.

23. The implantable apparatus of claim 1, wherein said remote electrical source comprises RF coupling means for transmitting electrical energy from the remote electrical source to an internal body cavity or vessel to be treated.

24. The implantable apparatus of claim 1, wherein said biocompatible matrix is placed on an external surface of an implantable placement substrate.

25. A method for the internal treatment of body cavities and damaged internal vessels using electromagnetic energy, comprising:
providing an illuminating device including a biocompatible matrix including an adhesive for attaching the reinforced matrix to a tissue surface to be treated, a plurality of electromagnetic energy sources mounted to said biocompatible matrix, and a remote electrical source in electrical communication with said plurality of electromagnetic energy sources for activation and control of energy emitted by said electromagnetic energy sources;
delivering the illuminating device to a body site to be treated;
applying the illuminating device to a tissue surface to be treated; and
irradiating the tissue surface;

26. The method of claim 25, wherein said step of delivering the illuminating device comprises introducing the illuminating device into a body cavity by an endoscopic procedure.

27. The method of claim 25, wherein said step of delivering the illuminating device comprises introducing the illuminating device into a body cavity by an endovascular placement system.

28. The method of claim 27, wherein said step of introducing the illuminating device into a body cavity by an endovascular placement system comprises mounting the illuminating device on an exterior surface of an endovascular placement system.

29. The method of claim 28, wherein said endovascular placement system comprises a stent.

30. The method of claim 28, further comprising the step of remotely controlling the remote electrical source by electrical connections to the remote electrical source.

31. The method of claim 28, further comprising the step of remotely controlling the remote electrical source by an RF coupling from an external source.

32. The method of claim 25, further comprising varying a radiation therapy.

33. The method of claim 25, further comprising varying a radiation frequency.

34. The method of claim 25, further comprising varying a surface treatment by time phasing of the energy from the light source.
